(19) United States
(12) Patent Application Publication
(10) Pub. No.: US 2020/0286592 A1
(43) Pub. Date: Sep. 10, 2020

(54) ANTIMICROBIAL SUSCEPTIBILITY TESTING USING LIBRARY MATCH

(71) Applicant: Highland Innovations Inc., West New York, NJ (US)

(72) Inventor: Eung Joon Jo, West New York, NJ (US)

(73) Assignee: Highland Innovations Inc., West New York, NJ (US)

(21) Appl. No.: 16/810,840

(22) Filed: Mar. 6, 2020

Related U.S. Application Data
(60) Provisional application No. 62/815,002, filed on Mar. 7, 2019.

(51) Int. Cl. G16B 40/10; G06N 5/04; G06N 5/02; G06N 20/00

(52) U.S. Cl. CPC G16B 40/10; G06N 20/00; G06N 5/02; G06N 5/04

(57) ABSTRACT

A method, apparatus, and/or computer program product that matches a treatment profile (e.g. drug treatment at a particular dosage and timeframe) to a sample of biological material (e.g. blood sample, etc.). A mass spectrometry spectrum may be generated from a matrix-assisted laser desorption ionization time of flight mass spectrometer (MALDI-TOF MS) device from the testing of a sample of biological material that includes at least one pathogen (e.g. bacteria, microorganism, virus, etc.). This received mass spectrometry spectrum may be matched by a diagnostic unit to determine the species of the pathogen against a species database. The species database may include predetermined mass spectrometry spectrums of known species of pathogens. Based on the matched species of pathogen, a diagnostic unit may match the received mass spectrometry spectrum of the sample of biological material to at least one treatment profile from a second reference database of predetermined mass spectrometry spectrums.

The present application claims priority to U.S. Provisional Patent Application No. 62/815,002 filed on Mar. 7, 2019, which is hereby incorporated by reference in its entirety.

### BACKGROUND

Illness of humans, animals, plants, and other living organisms is a natural part of life. It is naturally advantageous to minimize or cure illnesses to preserve or extend life. This is particularly true for human being's natural desire to preserve and extent their lives. Unfortunately, there are many unknowns in illnesses. Many illnesses are caused by pathogens, such as bacteria, viruses, microorganisms, etc., for which there are an uncountable number of species and strains. Not only is it difficult to detect a pathogen that has infected a human, but once the pathogen is identified, it is difficult to match the correct treatment (application of antipathogens). For example, bacteria may infect human beings and be treated with different antimicrobials. However, matching the right antimicrobial to the infecting bacteria may be especially challenging, since there are so many different strains of bacteria and humans can develop an immunity or resistance to different antimicrobial treatments. Further, there is time sensitivity to matching the right antipathogen treatment to a pathogen, as it is naturally desirable for the human to be cured as soon as possible from illnesses.

### SUMMARY

Embodiments relate to a method, apparatus, and/or computer program product that matches a treatment profile (e.g. drug treatment at a particular dosage and timeframe) to a sample of biological material (e.g. blood sample, etc.). In embodiments, a mass spectrometry spectrum may be generated from a matrix-assisted laser desorption ionization time of flight mass spectrometer (MALDI-TOF MS) device from the testing of a sample of biological material that includes at least one pathogen (e.g. bacteria, microorganism, virus, etc.). This received mass spectrometry spectrum may be matched by a diagnostic unit to determine the species of the pathogen (e.g. identification of a bacteria, microorganism, virus, etc.) against a species database. The species database may include predetermined mass spectrometry spectrums of known species of pathogens. Based on the matched species of pathogen, a diagnostic unit may match the received mass spectrometry spectrum of the sample of biological material to at least one treatment profile from a second reference database of predetermined mass spectrometry spectrums. In embodiments, since this diagnosis may be implemented based on innovative database matching techniques, the identification of treatments may be enhanced and the amount of time for diagnosis may be minimized.

### DRAWINGS

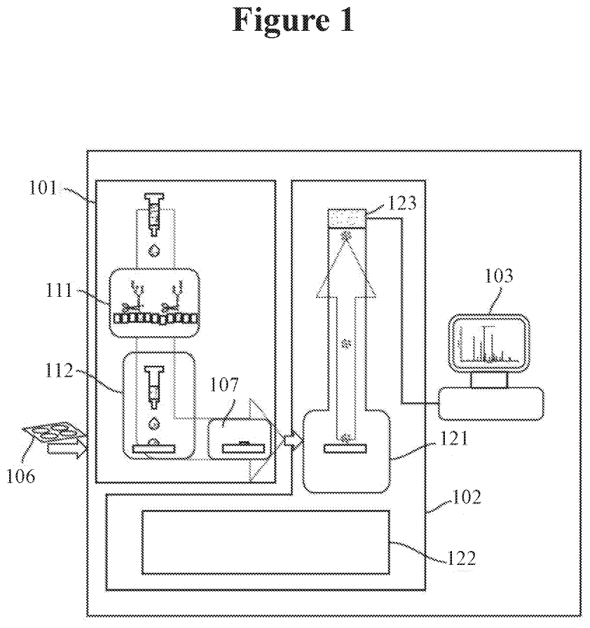

Example FIG. 1 illustrates an integrated disease diagnosis system, in accordance with embodiments.

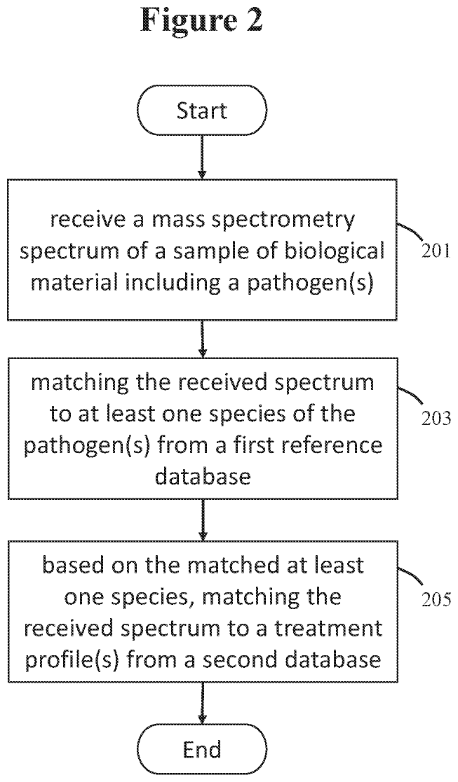

Example FIG. 2 illustrates a flow chart of matching a received mass spectrometry spectrum to a species database and a treatment profile database, in accordance with embodiments.

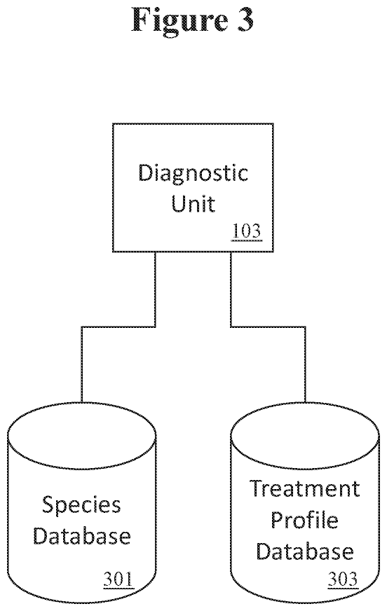

Example FIG. 3 illustrates a relationship between a diagnostic unit and a species database and a treatment profile database, in accordance with embodiments.

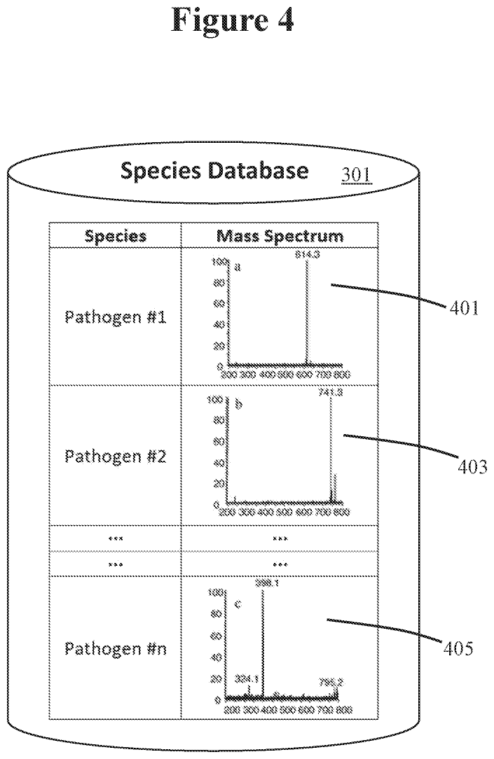

Example FIG. 4 illustrates a species database, in accordance with embodiments.

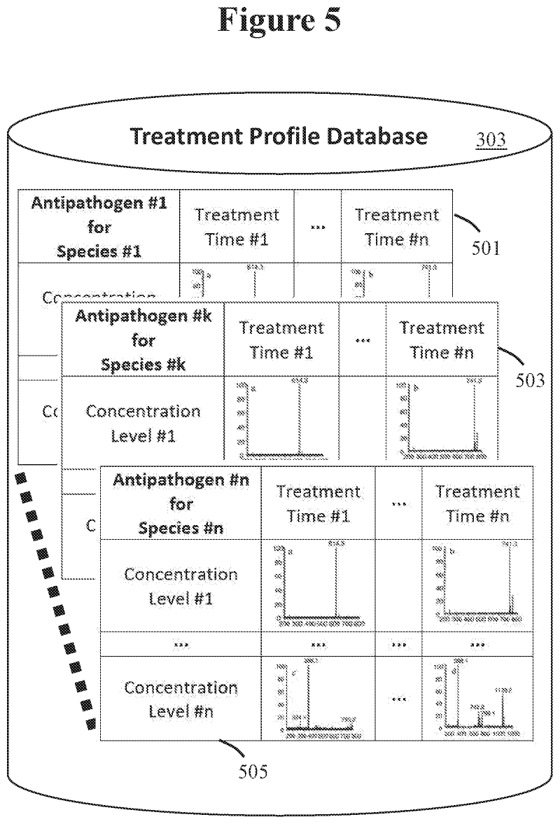

Example FIG. 5 illustrates a treatment profile database, in accordance with embodiments.

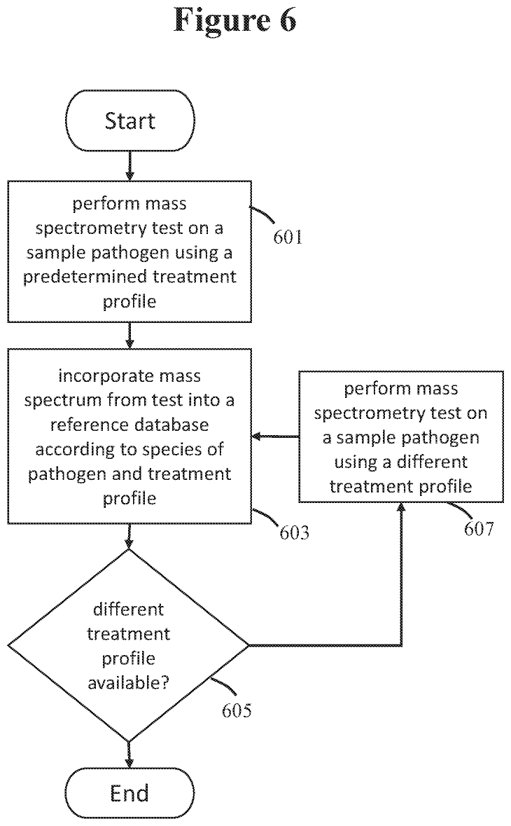

Example FIG. 6 illustrates a flowchart of constructing a treatment profile database, in accordance with embodiments.

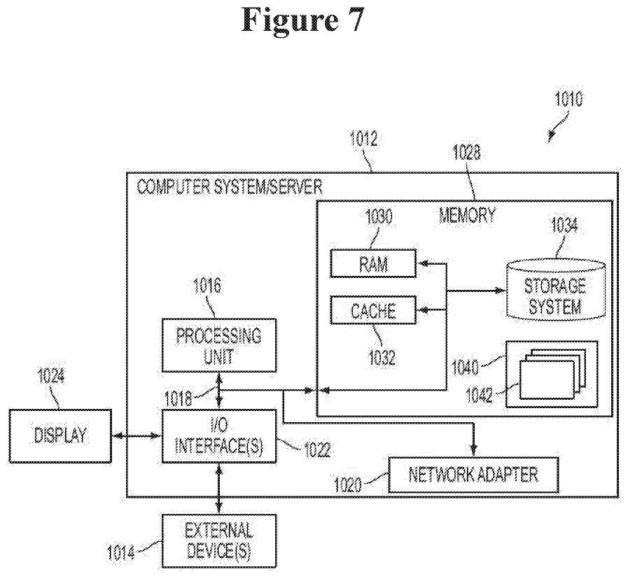

Example FIG. 7 illustrates a cloud computing node, in accordance with embodiments.

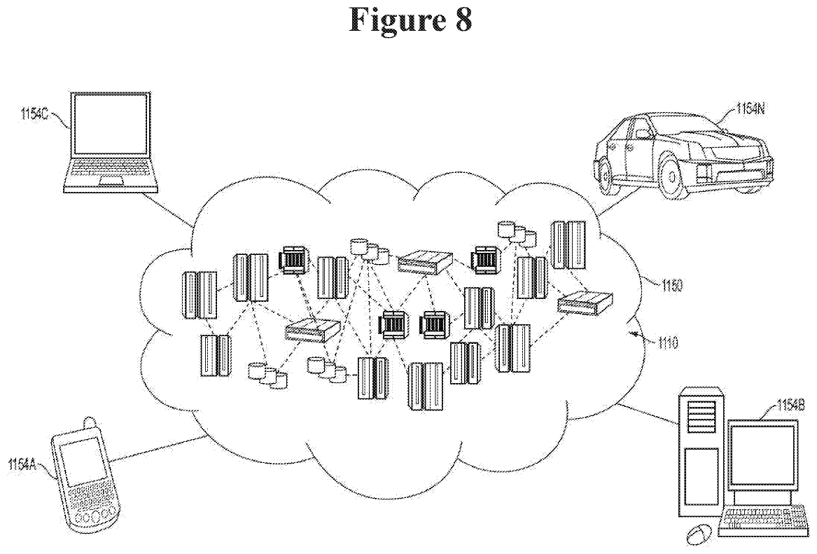

Example FIG. 8 illustrates a cloud computing environment, in accordance with embodiments.

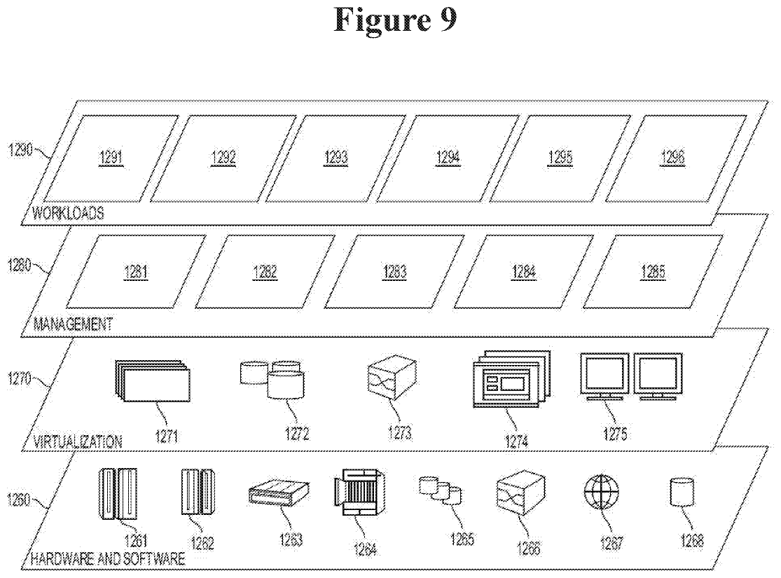

Example FIG. 9 illustrates abstraction model layers, in accordance with embodiments.

### DESCRIPTION

Example FIG. 1 illustrates an integrated disease diagnosis system, in accordance with embodiments. Samples (e.g. human samples containing pathogens) may undergo a combination of processes by selected modules. In the sample preparation system 101, a sample may go through a predefined and preprogrammed sequence depending on diagnosis or screening purposes in an automatic sample preparation unit 111, in accordance with embodiments. In embodiments, for glycan extraction, multiple processing modules may be selected, such as sample reception, protein denaturation, deglycosylation, protein removal, drying, centrifugation, solid phase extraction, spotting, and/or other processing. After sample preparation, the sample loader 112 may load the samples onto the plates 106 and are dried in a sample dryer 107, in accordance with embodiments. In embodiments, sample preparation unit 111 may not be included.

In embodiments, samples may then be provided to a MALDI-TOF MS unit 102 having an ion flight chamber 121 and/or a high voltage vacuum generator 122, in accordance with embodiments. A processing unit 123 in the MALDI-TOF MS may identify the mass/charge and its corresponding intensity. For the disease diagnostic purpose, those acquired mass and intensity data may be reorganized to set up a standard mass list, in which a concept of the center of mass where intensities are balanced and equilibrated is introduced. A standard mass to charge list is defined based upon the machine accuracy and the center of mass concept. The stored spectrum data (e.g. a mass spectrum) for each laser irradiation may also be used to set up the standard mass list.

In embodiments, diagnostic unit 103 may then compare, the spectra from a patient's sample with the pre-stored spectra and analyzes the pattern differences. The diagnostic unit 103 may then identify the presence and progress of the disease or pathogen. In embodiments, as shown in example FIG. 1, diagnostic unit 103 may be internally integrated to the MALDI-TOF MS unit 102. In embodiments, diagnostic unit 103 may be either internal or external to a mass spectrometer system. In embodiments, a diagnostic unit 103 may be cloud based. In embodiments, diagnostic unit 103 may employ artificial intelligence, machine learning algorithms, and/or deep learning algorithms. In embodiments, a diagnostic unit 103 may be networked to a mass spectrometer system by a local network (e.g. an intranet network), a public network (e.g. the internet), or any other network as appreciated by those skilled in the art. In embodiments, a diagnostic unit 103 may be coupled to an artificial intelligence engine and/or to one or more processors that implement machine learning and/or deep learning algorithms.

Example FIG. 2 illustrates a flow chart of matching a receive mass spectrometry spectrum to a first database and a second database, in accordance with embodiments. Embodiments relate to a method, an apparatus configured to implement the method, and/or a computer program products embodying the method. In step 201, a mass spectrometry spectrum of a sample of biological material including at least one pathogen may be received. In embodiments, the pathogen may be bacteria, a microorganism, a virus, and/or any other pathogen.

In step 203, the received mass spectrometry spectrum may be matched to at least one species of the at least one pathogen from a first reference database of predetermined mass spectrometry spectrums, in accordance with embodiments. In embodiments, the first reference database may be species database 301 illustrated in FIGS. 3 and 4. An unknown pathogen in the sample that undergoes processing by MALDI-TOF MS unit 102 may be identified by comparing the received mass spectrum to a first database that contains predetermined mass spectrums of already identified pathogens. Based on the similarity of the received mass spectrum of the sample containing an unknown pathogen and the first database that contains predetermined mass spectrums of known pathogen species, a species of the pathogen in the sample may be identified in step 203.

In step 205, based on the matched at least one species, the received mass spectrometry spectrum may be matched to at least one treatment profile from a second reference database of predetermined mass spectrometry spectrums. In embodiments, the second reference database is a treatment profile database 303 illustrated in FIGS. 3 and 5. Treatment profile database 303 may be relatively large, which may increase the chances of a false positive match. In step 205, by only matching the received mass spectrometry spectrum to treatment profiles stored in the second reference database that are known to be associated with the species of the pathogen contained in the sample (as determined in step 203), the treatment profiles stored in the second database are narrowed, in accordance with embodiments. In embodiments, by narrowing the treatment profiles stored in the second database, the chances of a false positive match are minimized and/or the chances of identifying the best possible treatment profile are maximized.

In embodiments, receiving the mass spectrometry spectrum includes receiving a mass-intensity profile of at least one pathogen from a sample. As may be appreciated by one of ordinary skill in the art, a sample may include one pathogen or a plurality of pathogens. In the case of a plurality of pathogens, then pathogens may be related and/or complementary, thus cumulatively resulting in a single mass spectrometry spectrum. A plurality of pathogens may be cumulatively be treated according to one treatment profile.

In embodiments, the sample includes biological material from a human being. In embodiments, the sample includes biological material from a living organism. In embodiments, the sample includes blood. In embodiments, the sample includes saliva. In embodiments, the sample includes feces. In embodiments, the sample includes urine. In embodiments, the sample includes organ tissue. In embodiments, the sample includes plasma. In embodiments, the sample includes skin. In embodiments, the sample includes hair. One of ordinary skill in the art would appreciate that the sample could contain any substance that can contain at least one pathogen.

In embodiments, at least one pathogen is a microorganism. Examples of microorganisms are bacteria, fungi, and mycobacteria. In embodiments, at least one pathogen is a virus.

In embodiments, the procedure illustrated in FIG. 2 may be implemented by using machine learning and/or artificial intelligence. In embodiments, the procedure illustrated in FIG. 2 may be performed in a cloud computing environment.

Example FIG. 3 illustrates a relationship between diagnostic unit 103 with a species database 301 and/or a treatment profile database 303, in accordance with embodiments. In embodiments, diagnostic unit 103 may have computational capacities which are able to compare and/or match similarities between a received mass spectrometry spectrum from a sample under test with predetermined reference mass spectrometry spectrums. Diagnostic unit 103 may be coupled to species database 301 with the ability to search, compare, and/or match the received mass spectrometry spectrum with the predetermined mass spectrometry spectrums stored in species database 301. Since the predetermined mass spectrometry spectrums stored in species database 301 include metadata that links them to species of pathogens, matching the received mass spectrometry spectrum to the data stored in the species database 301 allows the diagnostic unit 103 to identify the species of the pathogen (e.g. step 203 of FIG. 2).

Diagnostic unit 103 may also be couple to treatment profile database 303 with the ability to search, compare, and/or match the received mass spectrometry spectrum with the predetermined mass spectrometry spectrums stored in the treatment profile database 303. Since the predetermined mass spectrometry spectrums stored in the treatment profile database 303 include metadata that links them to specific treatment profiles of the already identified species of pathogens, matching the received mass spectrometry spectrum to the data stored in the treatment profile database 303 allows diagnostic unit 103 to identify at least one compatible treatment profile associated with the pathogen or pathogens contained in the sample.

Example FIG. 4 illustrates a species database 301, in accordance with embodiments. In embodiments, species database 301 includes a plurality of predetermined mass spectrometry spectrums (e.g. spectrums 401, 403, 405) which are each associated with species identifications of different pathogens (e.g. Pathogen #1, Pathogen #2, Pathogen # n, etc.). Diagnostic unit 103 may search, compare, and/or match the received mass spectrometry spectrum from the sample with the predetermined mass spectrometry spectrums (e.g. spectrums 401, 403, 405) in species database in order to identify and associate the pathogen contained in the sample to be used in future processing. The predetermined mass spectrometry spectrums (e.g. spectrums 401, 403, 405) may have been generated from previous tests where a specific pathogen species is accurately correlated to an associated mass spectrometry spectrum (e.g. Pathogen #1 may be correlated with mass spectrum 401). In embodiments, the accuracy in the identification of a species of a pathogen may be relatively high, so the accuracy of the matching results between the received mass spectrometry spectrum of the sample to the species database 301 may be relatively high.

Example FIG. 5 illustrates treatment profile database 303, in accordance with embodiments. In embodiments, treatment profile database 303 includes a plurality of mass spectrometry spectrums arranged in a plurality of tables (e.g. table 501, table 503, table 505, etc.). Each table 501, 503, 505 may be associated with a species of the pathogen determined in step 203 illustrated in FIG. 2. Since there may be multiple strains of species of the pathogens associated to treatment profile database 303, there will be multiple tables for each species of pathogen. In embodiments, each table 501, 503, 505 is associated with a particular strain of species of pathogen and a particular antipathogen (e.g. drug or drug treatment). In embodiments, there may be dozens, hundreds, or thousands of tables included in treatment profile database 303. When diagnostic unit 103 searches treatment profile database 303, only those tables associated with the species of pathogen which were previously identified (in step 203 in FIG. 2) are searched, thus minimizing the chances of a false positive result, in accordance with embodiments. For illustrative purposes, only three tables 501, 503, 505 are shown.

For example, table 505 is associated with antipathogen # n for species # n of a pathogen, in accordance with embodiments. There may be multiple tables also associated with antipathogen # n for species # n of a pathogen, with each of these tables being associated with a strain of species # n, in accordance with embodiments. Different strains of a pathogen may have differences which require different treatments, so it may be necessary to have different tables associated with each strain. In embodiments, treatment profile database 303 may not include a table for all strains of a pathogen for either practical or empirical reasons.

Each table of treatment profile database 303 includes variations of treatment of a strain of a pathogen to a particular antipathogen, in accordance with embodiments. In embodiments, these variations include a concentration level of an antipathogen and the amount of time the anti pathogen is applied to the reference pathogen (having a known species). Empirically, the best and/or ideal treatment profile can be determined by diagnostic unit 103 by matching the received mass spectrometry spectrum from a MALDI-TOF MS unit to the predetermined mass spectrometry profiles stored in treatment profile database 303. Given the potentially large number of mass spectrometry spectrums stored in treatment profile database 303, the parsing of treatment profile database 303 based on the known species of the pathogen contained in the sample reduces the chances false positive test results. In embodiments, it is irrelevant if the pathogen contained a sample that has undergone no treatment, since the empirical matching of the predetermined mass spectrometry spectrums may represent different stages of a strain of a pathogen reaction to a predetermined drug treatment (e.g. drug type, drug concentration, drug application time, etc.).

In embodiments, a plurality of treatment profiles may be stored in treatment profile database 303. Treatment profile database 303 may include a treatment drug applied to a matched species of at least one pathogen, in accordance with embodiments. Treatment profile database 303 may include an exposure time of a treatment drug applied to the matched species of at least one pathogen, in accordance with embodiments. Treatment profile database 303 may include a concentration of a treatment drug applied to a matched species of at least one pathogen, in accordance with embodiments.

In embodiments, at least one treatment profile may be correlated to at least one strain type of at least one species of at least one pathogen. In embodiments, matching a received mass spectrometry spectrum to at least one treatment profile from a treatment profile database 303 includes predicting susceptibility of at least one antipathogen to the at least one pathogen. In embodiments, the predicting susceptibility of the at least one antipathogen to the at least one pathogen includes predicting resistance of the at least one pathogen to the at least one antipathogen.

In embodiments, predetermined mass spectrometry spectrums of treatment profile database 303 is correlated with a reference antipathogen. In embodiments, predetermined mass spectrometry spectrums of treatment profile database 303 is correlated with an exposure time of a reference antipathogen. In embodiments, predetermined mass spectrometry spectrums of treatment profile database 303 is correlated with a concentration of a reference antipathogen. In embodiments, predetermined mass spectrometry spectrums of treatment profile database 303 is correlated with at least one matched species.

In embodiments, treatment profile database 303 includes a plurality of tables (e.g. 501, 503, 505, etc.). At least one of the pluralities of tables (e.g. 501, 503, 505, etc.) is associated with a strain type of the at least one pathogen and a reference antipathogen, in accordance with embodiments. Each entry of at least one of the plurality of tables (e.g. 501, 503, 505, etc.) includes a reference mass spectrometry profile associated with a strain type of the at least one pathogen response to a concentration of a reference antipathogen and an exposure time to the reference antipathogen.

In embodiments, each entry of at least one of the plurality of tables (e.g. 501, 503, 505, etc.) includes empirical data from previous tests of a strain type to a reference antipathogen. In embodiments, a strain type of at least one pathogen may be determined by comparing a received mass spectrometry spectrum to each entry of at least one of the plurality of tables (e.g. 501, 503, 505, etc.) and determining which entry is most similar to the received mass spectrometry spectrum.

In embodiments, an entry of at least one of the plurality of tables (e.g. 501, 503, 505, etc.) that is most similar to the received mass spectrometry spectrum identifies the strain type of at least one pathogen contained in a sample. In embodiments, an entry of at least one of the plurality of tables (e.g. 501, 503, 505, etc.) that is most similar to the received mass spectrometry spectrum identifies a recommended treatment profile for at least one pathogen contained in a sample. In embodiments, matching a received mass spectrometry spectrum to at least one treatment profile is agnostic to a strain type of the at least one pathogen contained in a sample.

Example FIG. 6 illustrates a flowchart of constructing a treatment profile database, in accordance with embodiments. In step 601, at least one mass spectrometry test may be performed on a sample of at least one pathogen to produce at least one mass spectrometry spectrum, in accordance with embodiments. In embodiments, at least one test on a sample may be perform after a predetermined treatment of the sample. In step 603, at least one mass spectrometry spectrum from a mass spectrometry test may be incorporated into a treatment profile database 303 (e.g. reference database). In embodiments, during the construction of treatment profile database 303, steps 605 and 607 may perform multiple tests to generate multiple tables (e.g. tables 501, 503, 505) in treatment profile database 303.

For example, in step 605, after step 603, it may be determined if there is a different treatment profile to be performed on the sample. If yes, then in step 607 a different treatment profile is mass spectrometry tested and the mass spectrometry results are incorporated into treatment profile database 303 in step 603. If no (in step 605), then the construction and incorporation of data into treatment profile database 303 for a particular sample is terminated.

In embodiments, a predetermined treatment may be at least one drug applied to a sample. In embodiments, a predetermined treatment may be a predetermined concentration of at least one drug applied to a sample. In embodiments, a predetermined treatment may be a duration of time that a predetermined concentration of at least one drug is applied to a sample.

In embodiments, at least one mass spectrometry spectrum includes a first set of mass spectrometry spectrums and a second set of mass spectrometry spectrums. A predetermined treatment associated with the first set of mass spectrometry spectrums may include application of a first drug treatment. A predetermined treatment associated with the second set of mass spectrometry spectrums may include application of a second drug treatment. The first drug treatment and the second drug treatment are different.

In embodiments, a first set of mass spectrometry spectrums is organized in a first table 501 and a second set of mass spectrometry spectrums is organized in a second table 502 or 503. Both the first table 501 and the second table 502 or 503 are incorporated into the treatment profile database 303.

In embodiments, a species of at least one pathogen is determined and associated with at least one mass spectrometry spectrum. The determining the species of the at least one pathogen includes comparing each of the mass spectrometry spectrums to a pathogen species database 301.

In embodiments, a sample includes at least one strain of at least one pathogen. A treatment profile database 303 (e.g. reference database) may include mass spectrometry data from a plurality of strains of the at least one pathogen, in accordance with embodiments. In embodiments, a treatment profile database 303 (e.g. reference database) may include mass spectrometry data from a plurality of pathogens. In embodiments, a treatment profile database 303 (e.g. reference database) may be used to identify a treatment profile of a patient infected by the at least pathogen. In embodiments, the procedure illustrated in FIG. 6 may be implemented by using machine learning and/or artificial intelligence. In embodiments, the procedure illustrated in FIG. 6 may be performed in a cloud computing environment.

Embodiments may be implemented in a cloud environment, as disclosed in U.S. patent application Ser. No. 15/682,251, filed on Aug. 21, 2017, which is incorporated by reference in its entirety.

Example FIG. 7 is a schematic of an example of a cloud computing node is shown. Cloud computing node 1010 is only one example of a suitable cloud computing node and is not intended to suggest any limitation as to the scope of use or functionality of embodiments of the invention described herein. Regardless, cloud computing node 1010 is capable of being implemented and/or performing any of the functionality set forth hereinabove.

In cloud computing node 1010 there is a computer system/server 1012, which is operational with numerous other general purpose or special purpose computing system environments or configurations. Examples of well-known computing systems, environments, and/or configurations that may be suitable for use with computer system/server 1012 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 1012 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 1012 may be practiced in distributed cloud computing environments where tasks are performed by remote processing devices that are linked through a communications network. In a distributed cloud computing environment, program modules may be located in both local and remote computer system storage media including memory storage devices.

Example FIG. 7 illustrates a computer system/server 1012 in cloud computing node 1010 is shown in the form of a general-purpose computing device. The components of computer system/server 1012 may include, but are not limited to, one or more processors or processing units 1016, a system memory 1028, and a bus 1018 that couples various system components including system memory 1028 to processor 1016.

Computer system/server 1012 may include a variety of computer system readable media. Such media may be any available media that is accessible by computer system/server 1012, and it includes both volatile and non-volatile media, removable and non-removable media. System memory 1028 can include computer system readable media in the form of volatile memory, such as random access memory (RAM) 1030 and/or cache memory 1032. Computer system/server 1012 may further include other removable/non-removable, volatile/non-volatile computer system storage media. By way of example only, storage system 1034 can be provided for reading from and writing to a non-removable, non-volatile magnetic media (not shown and typically called a “hard drive”).

Program/utility 1040, having a set (at least one) of program modules 1042, may be stored in memory 1028 by way of example, and not limitation, as well as an operating system, one or more application programs, other program modules, and program data. Each of the operating system, one or more application programs, other program modules, and program data or some combination thereof, may include an implementation of a networking environment. Program modules 1042 generally carry out the functions and/or methodologies of embodiments of the invention as described herein.

Computer system/server 1012 may also communicate with one or more external devices 14 such as a keyboard, a pointing device, a display 1024, etc.; one or more devices that enable a user to interact with computer system/server 1012; and/or any devices (e.g., network card, modem, etc.) that enable computer system/server 1012 to communicate with one or more other computing devices. Such communication can occur via Input/output (I/O) interfaces 1022. Still yet, computer system/server 1012 can communicate with one or more networks such as a local area network (LAN), a general wide area network (WAN), and/or a public network (e.g., the Internet) via network adapter 1020. As depicted, network adapter 1020 communicates with the other components of computer system/server 1012 via bus 1018. It should be understood that although not shown, other hardware and/or software components could be used in conjunction with computer system/server 1012. Examples, include, but are not limited to: microcode, device drivers, redundant processing units, external disk drive arrays, RAID systems, tape drives, and data archival storage systems, etc.

Example FIG. 8 illustrates cloud computing environment 1150, in accordance with embodiments. As shown, cloud computing environment 1150 comprises one or more cloud computing nodes 1110 with which local computing devices used by cloud consumers, such as, for example, personal digital assistant (PDA) or cellular telephone MA, desktop computer MB, laptop computer MC, and/or automobile computer system MN may communicate. Nodes 1110 may communicate with one another. They may be grouped (not shown) physically or virtually, in one or more networks, such as Private, Community, Public, or Hybrid clouds as described hereinabove, or a combination thereof. This allows cloud computing environment 1150 to offer infrastructure, platforms and/or software as services for which a cloud consumer does not need to maintain resources on a local computing device. It is understood that the types of computing devices MA-N shown in FIG. 8 are intended to be illustrative only and that computing nodes 1110 and cloud computing environment 1150 can communicate with any type of computerized device over any type of network and/or network addressable connection (e.g., using a web browser).

Example FIG. 9 illustrates a set of functional abstraction layers provided by cloud computing environment 1150, in accordance with embodiments. It should be understood in advance that the components, layers, and functions shown in FIG. 9 are intended to be illustrative only and embodiments of the invention are not limited thereto. As depicted, the following layers and corresponding functions are provided:

Hardware and software layer 1260 includes hardware and software components. Examples of hardware components include: mainframes 1261, RISC (Reduced Instruction Set Computer) architecture based servers 1262; servers 1263; blade servers 1264; storage devices 1265; and networks and networking components 1266. In some embodiments, software components include network application server software 1267 and database software 1268.

Virtualization layer 1270 provides an abstraction layer from which the following examples of virtual entities may be provided: virtual servers 1271; virtual storage 1272; virtual networks 1273, including virtual private networks; virtual applications and operating systems 1274; and virtual clients 1275.

In one example, management layer 1280 may provide the functions described below. Resource provisioning 1281 provides dynamic procurement of computing resources and other resources that are utilized to perform tasks within the cloud computing environment. Metering and Pricing 1282 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1283 provides access to the cloud computing environment for consumers and system administrators. Service level management 1284 provides cloud computing resource allocation and management such that required service levels are met. Service Level Agreement (SLA) planning and fulfillments 1285 provide pre-arrangement for, and procurement of, cloud computing resources for which a future requirement is anticipated in accordance with an SLA.

Workloads layer 1290 provides examples of functionality for which the cloud computing environment may be utilized. Examples of workloads and functions which may be provided from this layer include: mapping and navigation 1291; software development and lifecycle management 1292; virtual classroom education delivery 1293; data analytics processing 94; transaction processing 1295; and matching processing 1296 for spectrometer data.

In embodiments, when a patient is diagnosed with an infection of bacteria or fungi, he or she is treated with antimicrobials. An antibiotic drug, for example, prohibits the growth of bacteria. Some bacteria exhibit resistance to certain antibiotic agents. If the growth of a bacteria stain does not stop in the presence of antibiotics, the strain of the bacteria is resistant to the antibiotic agent. The resistance may be developed by gene mutation. The effectiveness may depend on the particular strain of a pathogen. A doctor may determine which antibiotic drug would be most effective in stopping the growth of the bacteria.

Antibiotic resistance presents many challenges, especially when monitoring and combating the infections of resistant bacteria. Examples of resistant bacteria include carbapenum-reistant enterobacetiacease (CRE), vacomycin-resistant enterococus (VRE), vancomycin-resistant Straphylococus aureus (VRSA), methicilin-reistant Straphylococus aureus (MRSA), mutidrug-resistant acinetogacter, multidrug-resistant Pseudomonias aeruginosa, and others.

An antimicrobial susceptibility test (AST) may be used to determine the effectiveness of particular antibiotics or antifungal and/or determine whether a strain of a bacteria has developed resistance to a specific antimicrobial agent, thereby helping the doctor select the most effective antimicrobial drug. AST may be done before an initial selection of antibiotic drugs or when the initial choice of antibiotic drug turned out to be ineffective.

In some applications of AST, pathogens are isolated from other microbes. The pathogens may be identified. Some pathogens are known to be susceptible to drugs, while other pathogens have unpredictable susceptibility to drugs. If an antibiotic drug is known to be effective to a pathogen, no further testing is necessary. If an antibiotic drug is known to be unpredictable to a pathogen, AST may be used to select the most effective antibiotic drugs.

There is a standard test called a disk diffusion test. A bacteria sample is placed in an agar plate. Antibiotic disks are placed on the surface. The growth of the bacteria sample is inhibited around each disk. The diameters of zone of inhibition are measured to determine susceptibility of each antibiotic agent.

One challenge of AST methods is that they may take several days to see the results. For example, it may take at least 24 hours to culture samples, another 24 hours to separate the pathogen, and yet another 24 hours to observe the response of stains against antibiotic over time. The amount of time and resources that AST methods can take may be a serious problem in life-threatening situation (e.g. such as sepsis) or other situations.

There some automated instruments to automate the AST processes. However, these automated processes still involve the time consuming process of administering different kinds of antibiotics to the samples each time when the susceptibility is to be determined.

There are gene-based methods to determine antibiotic susceptibility. For instance, methicillin-resistant Staphylcoccus aureus (MRSA) contains the mecA gene that confers resistance to the antibiotics methicillin, oxacillin, nafcillin, and dicloxacillin. Detection of the mecA gene using a molecular based test allows the rapid detection of MRSA prior to culturing the bacteria. However, gene-based methods may be limited to those pathogens with known mechanism as to which genes cause antibiotic resistance.

Embodiments relate to means to rapidly and accurately determine the susceptibility of antibiotic drugs without the need of applying antibiotic agents each time when the susceptibility is to be determined. Embodiments similarly relate to other pathogens, such as viruses.

Embodiments relate to use of a mass spectrometer, such as matrix-assisted laser desorption ionization time of flight mass spectrometer (MALDI-TOF MS). MALDI-TOF MS devices may use a soft ionization technique to determine mass information of a protein without destroying protein structure. MALDI-TOF MS devices may be a rapid and cost-effective analysis to identifying bacteria species compared to DNA-based analysis. MALDI-TOF MS may be used to identify bacteria, fungi, mycobacteria, and/or other substances.

Embodiments use a library-based approach of analyzing antimicrobial resistance of a new patient's sample without the process of experimenting with antibiotics at the time of diagnosis. Mass spectrums generated by MALDI-TOF MS devices may contain information on drug-resistant strains of pathogens, in accordance with embodiments. Mass spectrums may contain information of specific proteins that may make a particular antibacterial agent ineffective, in accordance with embodiments.

Embodiments use a mass spectrometer to construct a library of antibiotic susceptibility. The antibiotic susceptibility library is a database storing the information on how a particular bacteria subspecies type or strain type reacts to a certain antibiotic agent over time.

For example, there may be six different kinds of bacteria species A, B, C, D, E, and F that are of interest for an AST. Each of the bacteria species may have multiple types of strains associated with a particular bacteria. For example, species A may have 20 different strains (e.g. A1, A2, A3, . . . , A20). For example, there may be 10 different kinds of antibiotic candidates are available (‘a’, ‘b’, ‘c’, . . . , ‘j’) for these 20 different strains of species A.

Using a MALDI-TOF MS device, multiple tables may be constructed that serve as a susceptibility library to analyze the susceptibility of an antipathogen from a pathogen contained in a patient's sample, in accordance with embodiments. From this susceptibility library, in embodiments, a strain type of a species of pathogen in a patent's sample may be determined by analyzing a received sample mass spectrum with predetermined mass spectrometry spectrums stored in the susceptibility library. In embodiments, using the received mass spectrometry spectrum and comparing it to the susceptibility library, an ideal or preferred antibiotic agent that is most effective against a strain of pathogen may be determined. The tables may also identify the optimal concentration of antipathogen drug to be administered to stop the growth of a particular bacteria. In embodiments, the tables may be used without identification of the specific strain of the pathogen.

In embodiments, when a sample from a new patient is to be analyzed for antibiotic sensitivity, a blood sample may be obtained from a patient and cultured. A mass spectrometer, such as a MALDI-TOF MS device, may be used to obtain the mass-spectrum of the patient's sample, in accordance with embodiments. The obtained mass spectrum may be analyzed to determine a species on the basis of the susceptibility library, in accordance with embodiments. The patient's mass spectrum may be further analyzed with respect to the susceptibility reference database, in accordance with embodiments. In embodiments, by finding the closest match between a patient's mass spectrum and predetermined mass spectrum entries in a susceptibility library, the type of the strain may be determined, an effective antipathogen agent may be determined, and/or an optimal amount of a determined antipathogen agent to be administered may be determined.

In embodiments, analysis may involve comparing several significant peaks or peak patterns of a new sample mass spectrum against predetermined mass spectrums stored in a susceptibility library. Machine learning methods such as K-nearest neighbor (KNN), support vector machine (SVM), random forest, and/or other methods may be used to discriminate subtle spectral differences, in accordance with embodiments.

In embodiments, analysis may be supplemented by variations in the amount of antipathogens applied in the susceptibility library. For example, when a sample strain of a pathogen acquires resistance during interaction with antipathogens by gene mutation, it may no longer interact with the antipathogens, thereby consuming less of the antipathogen. The amount of antibiotic consumed may be an indication of having acquired resistance.

There are several example mechanisms how a microorganism may acquire resistance. The microorganism may change its composition by mutation to develop resistance. Such spectral changes of a sample strain may be detected by analyzing changes in mass spectrum of sample components.

Another example of how a microorganism acquires resistance to an antimicrobial agent is for a microorganism to generate an entity that destroys the effectiveness of an antimicrobial agent. For example, β-lactamase enzyme destroys the β-lactam core structure essential to antibiotics. The spectral changes of β-lactamase activity may be detected by analyzing changes in mass spectrum in the antibiotic components.

It will be obvious and apparent to those skilled in the art that various modifications and variations can be made in the embodiments disclosed. This, it is intended that the disclosed embodiments cover the obvious and apparent modifications and variations, provided that they are within the scope of the appended claims and their equivalents.

What is claimed is:

1. A method comprising:
receiving a mass spectrometry spectrum of a sample of biological material comprising at least one pathogen;
matching the received mass spectrometry spectrum to at least one species of the at least one pathogen from a first reference database of predetermined mass spectrometry spectrums; and
based on the matched at least one species, matching the received mass spectrometry spectrum to at least one treatment profile from a second reference database of predetermined mass spectrometry spectrums.

2. The method of claim 1, wherein the treatment profile from the second database comprises at least one of:
a treatment drug applied to the matched species of the at least one pathogen;
an exposure time of the treatment drug applied to the matched species of the at least one pathogen; or
a concentration of the treatment drug applied to the matched species of the at least one pathogen.

3. The method of claim 1, wherein the at least one treatment profile correlates to at least one strain type of the at least one species of the at least one pathogen.

4. The method of claim 1, wherein the at least one pathogen is a microorganism comprising at least one of bacteria, fungi, or mycobacteria.

5. The method of claim 1, wherein the matching the received mass spectrometry spectrum to the at least one treatment profile from the second database comprises predicting susceptibility of at least one antipathogen to the at least one pathogen.

6. The method of claim 5, wherein the predicting susceptibility of the at least one antipathogen to the at least one pathogen comprises predicting resistance of the at least one pathogen to the at least one antipathogen.

7. The method of claim 1, wherein at least one of the predetermined mass spectrometry spectrums of the second database are correlated with:
a reference antipathogen;
an exposure time of the reference antipathogen;
a concentration of the reference antipathogen; and
the matched at least one species.

8. The method of claim 7, wherein:
the second database comprises a plurality of tables;
at least one of the pluralities of tables is associated with a strain type of the at least one pathogen and the reference antipathogen; and
each entry of at least one of the plurality of tables comprises a reference mass spectrometry profile associated with the strain type of the at least one pathogen response to the concentration of the reference antipathogen and the exposure time to the reference antipathogen.

9. The method of claim 8, wherein the strain type of the at least one pathogen is determined by comparing the received mass spectrometry spectrum to the each entry of at least one of the plurality of tables and determining which entry is most similar to the received mass spectrometry spectrum.

10. The method of claim 9, wherein the entry that is most similar to the received mass spectrometry spectrum identifies at least one of:
the strain type of the at least one pathogen; or
a recommended treatment profile selected from the at least one treatment profile.

11. The method of claim 1, wherein the method comprises the use of at least one of machine learning or artificial intelligence.

12. The method of claim 1, wherein the method is performed in a cloud computing environment.

13. A method comprising:
performing at least one mass spectrometry test on a sample of at least one pathogen to produce at least one mass spectrometry spectrum, wherein the at least one test on the sample is perform after a predetermined treatment of the sample; and
incorporating the at least one mass spectrometry spectrum into a reference database.

14. The method of claim 13, wherein the predetermined treatment comprises at least one of:
at least one drug applied to the sample;
a predetermined concentration of the at least one drug applied to the sample; or
a duration of time that the predetermined concentration of the at least one drug is applied to the sample.

15. The method of claim 14, wherein:
the at least one pathogen comprises a microorganism; and
the at least one drug comprises an antibiotic.

16. The method of claim 13, wherein:
the at least one mass spectrometry spectrum comprises a first set of mass spectrometry spectrums and a second set of mass spectrometry spectrums;
the predetermined treatment associated with the first set of mass spectrometry spectrums comprises application of a first drug treatment;
the predetermined treatment associated with the second set of mass spectrometry spectrums comprises application of a second drug treatment; and
the first drug treatment is different from the second drug treatment.

17. The method of claim 16, wherein:
the first set of mass spectrometry spectrums is organized in a first table;
the second set of mass spectrometry spectrums is organized in a second table; and
both the first table and the second table are incorporated into the reference database.

18. The method of claim 13, comprising:
determining a species of the at least one pathogen; and
associating the species of the at least one pathogen to each of the at least one mass spectrometry spectrum.

19. The method of claim 13, wherein the method comprises the use of at least one of machine learning or artificial intelligence.

20. The method of claim 13, wherein the method is performed in a cloud computing environment.
